(19) United States
(12) Patent Application Publication
(10) Pub. No.: US 2022/0378662 A1
(43) Pub. Date: Dec. 1, 2022

(54) COMPLIANCE SYSTEM AND SMART PACKAGE SYSTEM FOR DYNAMICALLY DISPLAYING USE CONDITIONS

(71) Applicants: Michael PETERSEN, Ottawa (CA); Allan WILSON, Ottawa (CA); Dean BROTZEL, Ottawa (CA); INTELLIGENT DEVICES SEZC INC., GeorgeTown (KY)

(72) Inventors: Michael Petersen, Ottawa (CA); Allan Wilson, Ottawa (CA); Dean` Brotzel, Ottawa (CA)

(21) Appl. No.: 17/773,782

(22) PCT Filed: Oct. 30, 2020
(86) PCT No.: PCT/CA2020/051474

§ 371 Date: May 2, 2022

(30) Foreign Application Priority Data
Oct. 30, 2019 (CA) 3060303
Nov. 16, 2019 (CA) 3061876
Nov. 29, 2019 (CA) 3063247

(51) Int. Cl. A61J 7/04; G16H 20/10; A61J 1/18

(52) U.S. Cl. CPC A61J 7/0409; G16H 20/10; A61J 1/18; A61J 2200/30; A61J 2200/72; A61J 2205/60

(57) ABSTRACT

There is provided a system for monitoring a package. The system comprises a scanning device and a dynamic display connected to the package and comprising a display. The scanning device scans the dynamic display and sends the scanned results to a server, which processes the results and sends the scanning device an update for the dynamic display. The scanning device then sends the update to the dynamic display and the dynamic display changes the display to show the update. There is also provided a system for monitoring compliance of a user with health related activities, assisting a user with compliance, encouraging a user to comply and detecting compliance.

### TECHNICAL FIELD

There is provided a system for monitoring a package. More particularly, the system includes a dynamic display. There is also provided a system for monitoring compliance of a user with health related activities, assisting a user with compliance, encouraging a user to comply and detecting compliance.

### BACKGROUND

Devices for monitoring, recording and downloading medication compliance data for vials, bottles, syringes and blister packages are well known. Allan Wilson, Michael Petersen, Dean Brotzel, Jakob Ehrensvaerd and Stina Grip, amongst others, have described such devices for blister packaged medication, for example U.S. Pat. Nos. 7,113,101, 7,178,417, 6,628,199, 6,244,462, 7,170,409, 6,616,035, 7,616,116 and 7,772,974; PCT applications WO/2009/135283, and WO 2013/159198 A1; Canadian application No. 2353350 and US Publication Nos. 20070278285, 20080191174 and 20080053222. Such devices are commonly referred to as smart packages.

The pharmaceutical industry is required to advise patients regarding expiry of medication. Such information is usually provided on a label printed and put on the medication packaging or container, or the information can be printed directly onto the box or packaging for the medication. If any information on a label changes, the label cannot be reprinted and replaced easily. Regulations require the pharmaceutical industry to repackage the medication contents in new packaging. Oftentimes medication must be recalled to perform the repackaging if new information comes to light regarding anticipated expiry dates or other details. This causes delay and extra expense.

Beyond ensuring a patient has access to an appropriate and accurate expiry date, there are other aspects of patient compliance that can be challenging. Encouraging patients to adhere to medication dosage regimes can be difficult and ensuring that the medication dosage regimes are followed can be critical. Many patients take several medications each day, each medication having a different expiry date, different dosage regime and requiring different quantities. Furthermore, each medication often has dosage requirements, timing requirements, food intake requirements and other related elements, which can be difficult for a user to track. It is also difficult, if not nearly impossible for a user to track any changes to expiry, because only a pharmacy or doctor would know if medication that has been dispensed to a patient has undergone a change of expiry from the pharmaceutical provider. Furthermore, it would be impossible for anyone to be aware of a change in expiry date due to other reasons, such as environmental conditions.

### SUMMARY

The present disclosure provides, in one aspect, a novel ability to update the label on a pharmaceutical package without requiring repackaging of the contents in a new package and without requiring recalling the medication from distribution.

In one aspect, there is described herein a dynamic display which a user can scan with a scanning device. In one example embodiment, the dynamic display measures temperature and time and displays information to the user. The scanning device consults a cloud, server or other database. The scanning device retrieves the results from the server and optionally runs various processes. Alternatively, or in addition, the cloud or server or the dynamic display can run the processes. The scanning device sends an update to the dynamic display in order to change the information shown on the display.

In one example embodiment, the dynamic display can show or display various information such as expiration date, current time, current date, temperature information, time of next dose and a status indicator. Some or all of the information can be updated by the scanning device. Various colour schemes can be used to indicate different information.

In another further aspect, there is provided a compliance system, comprising a scanning device which receives one or more health related activities as an input from a user. In one example embodiment, the compliance system has a slider for allotting a time interval and a counter for allotting dosing frequency. A series of one or more clocks are connected to the slider and the counter wherein the clocks set the time during a predetermined time period for performing one of the health related activities based on the dosing frequency and time interval.

The compliance system can assist a user in scheduling all health aspects of their life, for example, but not limited to, exercise, food preparation, food consumption, meal times, medication dosage times, medication expiry, meditation, prayer, and other lifestyle related items.

### BRIEF DESCRIPTION OF THE DRAWINGS

The invention will be further understood from the following description with reference to the attached drawings.

FIG. 1 depicts an example medication package having a dynamic display showing example information thereon.

FIG. 2 depicts an example display of an example app or computing program on the scanning device and sample information that the scanning device can show, including temperature.

FIG. 3 depicts an example display of an example app or computing program on the scanning device and sample information that the scanning device can show, including a slider and a series of clocks.

### DETAILED DESCRIPTION OF PREFERRED EMBODIMENTS

The exemplary embodiments of the present disclosure are described and illustrated below to encompass a dynamic display and compliance system for example purposes only. It will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present disclosure.

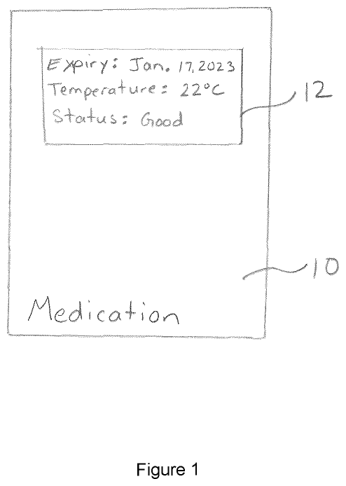

In one example embodiment, FIG. 1 shows a sample medication package 10 having a dynamic display 12 thereon. The medication package 10 could be a blister package, bottle, vial, syringe or any other type of medication package. The example dynamic display 12 shows example types of information, in this case showing the expiry date, temperature and status.

The dynamic display 12 can be formed from various methods. For example, the display portion can be printed with printable ink, optical ink or can be formed with any other suitable method of display. In one example embodiment, the dynamic display is printed with e-ink which has the advantage that the ink only requires power when the display changes. Once changed, the e-ink does not require power to maintain the changed display.

In another example embodiment, the dynamic display uses two colours of e-ink, for example green can illustrate good status such that the medication is viable and can be consumed, while red can indicate bad status such that the medication has been exposed to time or temperature combinations that dictate it should no longer be consumed. A third colour can be used to provide other information, such as expiry date. More colours can be used as necessary, for example to show temperature status or time of next dose.

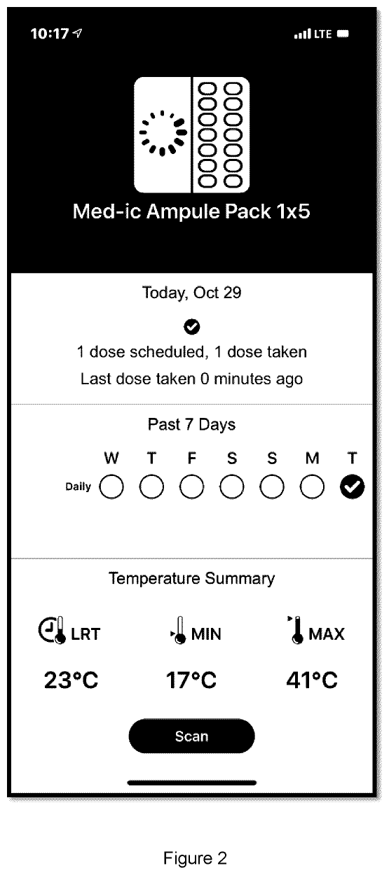

In another example embodiment, FIG. 2 shows an app on an example scanning device for receiving information from a dynamic display on a medication package. The scanning device and dynamic display communicate using any suitable form of communication, such as NFC (Near Field Communication). In cases where the scanning device does not have NFC, other methods can be used to communicate such as encoding dose information in a dynamic QR code (Quick Response Code) on the display for optical scanning by the scanning device.

In FIG. 2, the scanning device is shown as a smartphone, however the scanning device can be any example of hand-held scanner, such as an RFID tag reader, a smart phone, a tablet, or any other type of scanner.

In one example preferred embodiment, a smart phone has an app that is used by the user. The user scans the dynamic display and the smart phone communicates with the server or cloud to determine whether there are any updates to the information on the display. The smart phone then communicates with the dynamic display using NFC or other communication means to send the updated information to the dynamic display. The dynamic display then changes to show the updated information. The app on the smart phone can be used to optionally show the history and analytics of updated information, such as maximum and minimum temperatures, along with current information, such as current expiry date. The processes to determine if the display needs updating can be run by the server or cloud, or on the app on the smart phone or on the dynamic display itself.

In another example embodiment, the dynamic display tracks the temperature either continuously or periodically and stores the temperature internally in a storage medium for use by the dynamic display. In another embodiment, the dynamic display has a transmitter and receiver for transmitting the stored temperature data to the server, the scanning device or elsewhere. In another embodiment, the dynamic display polls the temperature periodically and transmits the result to the server, the scanning device or elsewhere. The transmission of the data can occur continuously or the data can be stored and transmitted when the scanning device comes into range.

In a preferred example embodiment, the scanning device is a smart phone and the dynamic display is operatively connected or integrated within a smart package or smart cap containing medication. The smart package has an RFID tag which communicates with the dynamic display. In such an example embodiment, the tag can contain the temperature sensor, time tracking and dose monitoring system and the dynamic display can display the information showing temperature, expiry, time, time of next dose or other information. Alternatively, the dynamic display can contain the temperature sensor and time tracking, in which case the dynamic display can optionally have an internal clock. Internal storage can be optionally provided on the dynamic display or the tag.

The processes that are run by the server, dynamic display or scanning device determine whether the combination of elapsed time and exposed temperature have led to conditions such that the medication should no longer be consumed. In one example embodiment, when the process is run on the server, the smart phone scans the dynamic display and an app on the smart phone communicates with the server. The server processes the data to determine if updates to the dynamic label are required. The app on the smart phone receives the results from the server and transmits the results to the dynamic display in order to update the information shown on the dynamic display. In this way, a user can determine at a glance whether the medication is consumable prior to opening the package and consuming the medication.

The app on the smart phone can be used to optionally show a history of updated information, such as maximum and minimum temperatures and/or a history of dosage regime, along with current information, such as current expiry date, current date, time and/or date of next dose, time and/or date of last dose, number of doses taken, number of doses scheduled for the next time period and/or amount of remaining medication.

As discussed above, the dynamic display can be updated based on information the scanning device finds in the cloud. In another variation, the dynamic display on a smart package or smart cap has predefined parameters which allow the temperature monitoring tag of the smart package to run a process and determine expiration conditions of the package contents. The expiration conditions or other details determined by the tag are then communicated from the tag to the dynamic display to change the information shown on the display without needing a scanning device to connect to a server.

In a further example embodiment, the dynamic display has its own transceiver (for example, Bluetooth or other over-the-air low power protocols) and periodically checks for updates directly from the server over communication channels. In other alternatives, updates can be automatically “pushed” to the dynamic display and/or the dynamic display can update the cloud or server data regarding the expiration status and/or use status that are determined autonomously by the dynamic display. The dynamic display device in one example embodiment has predetermined parameters upon which it can make autonomous expiration or best-used-by decisions and, without further external input, update the display.

The dynamic display has one advantage in that the dynamic display can be updated between doses. For example, a user could scan the dynamic display prior to consuming a first dose of medication and the results of the process could show a green status that the medication is good to consume. At the time of taking the next dose, the user can scan the dynamic display again and determine whether the next dose is good to consume. If conditions have changed between the time the first dose was taken and the second dose was scanned, it is possible the second dose could be bad to consume. As another alternative, once a first dose is scanned, the process might result in an updated expiry date on the dynamic display, showing that the remaining contents must be consumed within a shorter time frame in order to remain good to consume.

In a further example embodiment, the dynamic display system is able to calculate remaining best-used-by dates based on previous storage conditions and provide this information to the user, for example either on the package, the scanning device, the user's mobile phone or in the cloud/server. This would enable the user to determine if all of the package contents could be consumed prior to expiry.

In a further example embodiment, the scanning device has multiple profiles for registering multiple users or multiple medications. The scanning device is able to update each profile individually or collectively as a whole.

In another example embodiment, the dynamic display is a standalone sticker or has another method of attachment so the dynamic display can be attached, adhered or connected to the package which is being monitored. The sticker could have an optional feature of being reusable by not requiring permanent adhesive. Such an embodiment is useful in instances, for example, where compliance with medication consumption is not a priority, however the integrity of the package contents is important.

It could be beneficial to detect conditions at the time of removal of a dose. In one example embodiment, a smart package, smart cap or dynamic display has a button or other indicator which a patient would push or mark each time a dose is removed.

The button or indicator can be used by a patient to indicate the removal of a medicine vial from a temperature sensitive package, for example. The smart package, smart cap or dynamic display records the time and conditions of dosage removal, which is made available to the scanning device. As an alternative system, the button is provided on the scanning device so a user can mark off the administration of a dose, while the smart package, smart cap or dynamic display records the time and conditions of dosage removal. As a further alternative, the smart cap or smart blister package auto-records the removal of a dose, along with the time, temperature or other related environmental conditions at the time of removal. The smart cap, smart package or dynamic display transmits the information as per other methods discussed herein, such as storing the results in internal storage or communicating the results to the scanning device either at periodic intervals or only when scanned by the scanning device.

Once removal of a dose has been detected and the conditions have been recorded, the compliance system optionally determines whether the medicine dose was removed while the package was within allowable temperature limits and/or before the medication expired. A warning or alert could be generated if the timing of removal of the dose fails to meet safe consumption requirements. Either the smart package, smart cap, dynamic display, server or scanning device can determine whether the dose was safe and/or provide the warning or alert.

In a variant, the compliance system records the temperature history related to the removal of each dose from the package. Through tracking the history, the compliance system can determine and calculate the remaining best-used-by dates through use of predefined parameters.

It will be appreciated by one skilled in the art that variants can exist in the above-described arrangements and applications. For example, while this description has been limited to use of a dynamic display with medication packaging for example purposes, the compliance system and/or dynamic display could be used on alternative content packaging systems.

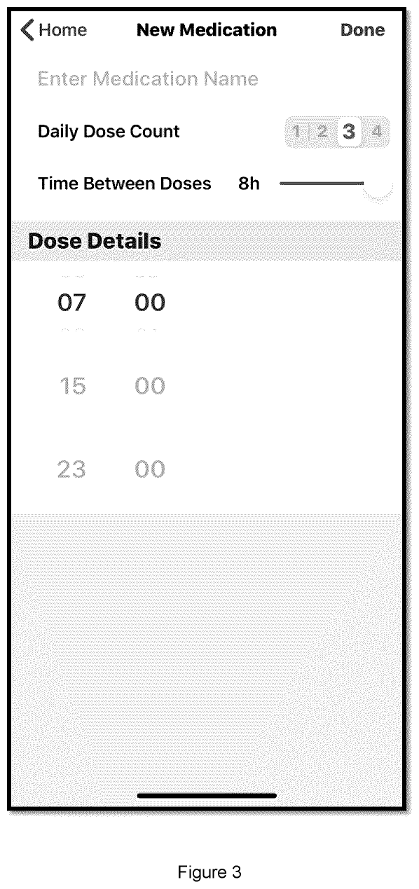

FIG. 3 shows an app on an example scanning device having a slider and a series of clocks to assist a user with compliance. In this example, by adjusting the number of dosages and moving the slider to indicate the spacing per dose, the user can control the times for taking the medication. The time period is usually a day, but it could be a different period of time, such as the span of multiple days, a week, a month or any other time period that is reasonable for the timing requirements. The app on the smart phone of FIG. 3 can be combined with the app of FIG. 2 so that a consumer has a comprehensive app for monitoring expiry dates and timing of health related activities.

In one example, the scanning device can be set up to avoid setting dosage times overnight when the user would be sleeping. As another example, if the user has an activity at a particular time during that day, say for example 2:00 pm, such that the user would not be able to take their medication at that time, the user can adjust the time slider until a better, more convenient, dosage time is obtained.

In FIG. 3, the scanning device is shown as a smartphone, however the scanning device can be any example of hand-held scanner, such as an RFID tag reader, a smart phone, a tablet, or any other type of scanner. Furthermore, the scanning device can operate with a smart phone or can alternatively be integrated into the smart phone. Thus, the slider and series of clocks can be provided on a separate device which communicates with the scanning device in any reasonable communication method, such as those discussed herein.

The compliance system permits the user to use a time slider device and a series of clocks that are based on the number of doses of medication required by the user in that time period. As the user adjusts the slider, each clock is adjusted to provide the user with the new times for taking the medication during that particular time period, based on the number of doses required during the time period and the slider time interval between doses. A user can adjust the number of doses and time interval. The user moves the slider to adjust the time between doses. By adjusting the time between doses, the user can control the times for taking the medication.

In another aspect, there is provided a compliance system that assists a user in setting up family health. The system can be used for multiple family members concurrently by setting up a different profile for each family member. In one example, the scanning device enables the user to set up, not only dosage times, but also times for other health related daily activities, such as exercise or movement activities. For example, a user might be required to walk for 10 minutes three times a day, or a physiotherapist might require a patient to practice standing 5 times a day. In one example, such repetitive daily activities can be entered into the scanning device and scheduled along with medication dosage times or other health related activities.

In another example, the compliance system can assist a user in scheduling all health aspects of their life, for example, but not limited to, exercise, food preparation, food consumption, meal times, medication dosage times, medication expiry, meditation, prayer, and other lifestyle related items. For example, this could be useful for a user who must schedule certain medications with meals and other medications away from meals, or for a user who must take certain medications at separate times throughout the day to reduce the likelihood of drug interactions.

In one example, the compliance system comprises a code on a package, wherein the scanning device scans the code, determines the contents of the package and inserts the type of contents as one of the health related activities in the scanning device. The package can have any type of contents related to health. As an example, the compliance system can operate in conjunction with a smart cap or smart blister package containing medication therein. The smart cap or blister package has a scan code associated with it, for example, a barcode, RFID chip, NFC or QR code. The user can scan the code with a scanning device. When determining the content of the package, the scanning device can optionally set the number of dosages and the spacing per dose.

In another aspect, there is provided a compliance system having an alarm system in which a user can receive reminders or alarms at the appropriate time for a health related activity. The reminders can be sent to the scanning device, to one or more cell phones via text message or SMS message, to the email address of the user, to a telephone call, or to any other manner of reminder system selected by the user. The alarm system can be set to send reminders.

In a further example aspect, use data from the compliance system and/or scanning device is sent to a physician, psychologist or other health practitioner in order to track the compliance of the user to the lifestyle requirements prescribed by the health practitioner. In order to communicate such information, the scanning device can have a transmitter and receiver for transmitting the compliance data periodically or continuously to a cloud, server or other database. The scanning device can optionally store the compliance data in internal storage memory temporarily or permanently. Alternatively, the scanning device might not have internal storage and instead, the scanning device might be separate from the compliance system in which case, the scanning device can periodically poll the compliance system and transmit the results to the cloud, server or other database or administrator without storage. Alternatively, the user can mark or indicate completion of each health-related activity with an indicator on the system or the scanning device. The indicator sends the completed health-related activities to a health practitioner in order to track the compliance of the user to the lifestyle requirements prescribed by the health practitioner.

In another aspect, there is a provided a compliance system in which the user receives rewards for good compliance by tracking the compliance of the user and using behaviour modification through a reward system to increase compliance through provision of rewards to the user. In one example, the reward system could comprise either positive or negative reinforcement to encourage the user to comply with the dosage regime or other health related activities. For example, negative reinforcement could include payment by the user of a sum of money in advance after which the user receives a set amount of money returned for compliance, such as for each pound or for pain reduction due to weight loss.

In one example, such a reward system can be tracked and implemented by compliance system, including the scanning device and/or through communication from the scanning device to the cloud, server, database or other administrator. In this example, the reward system encourages the user to comply with the dosage regime or other health related activities. The reward system could involve coupons from stores at which the user generally shops. Other alternatives for a reward system include, but are not limited to, discounts, credits towards future purchases, or other monetary forms of reward.

In one example involving various aspects, the health practitioner can set up a schedule on the compliance system for one or more health related activities for the user. The health practitioner could have the ability to lock the schedule in part or in full so that the user receives the completed schedule or so the user is able to set the times of use each day through the slider device. Alternatively, the user could enter all health related activities into the compliance system. As a further alternative, the user could scan the smart package or smart cap, which enables the scanning device to enter the medication into the compliance system, with proposed or fixed dosage regimes. When the user completes a scheduled health related activity or task, the user can mark or indicate completion through a push button or other indicator and the compliance system can record the time of completion. As the user increases compliance, the compliance system can provide the user with rewards and incentives, which will further encourage the user to increase compliance.

The compliance system can enable the scanning device to be updated based on information the scanner receives from the cloud, server, database or other administrator, such as the health practitioner.

Following from the above description, it should be apparent to those of ordinary skill in the art that, while the methods and apparatuses herein described constitute exemplary embodiments of the present invention, the invention described herein is not limited to any precise embodiment and that changes may be made to such embodiments without departing from the scope of the invention as defined by the claims. Consequently, the scope of the claims should not be limited by the preferred embodiments set forth in the examples but should be given the broadest interpretation consistent with the description as a whole. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

1. A compliance system, comprising:
a dynamic display on a package, the dynamic display comprising an adjustable display for providing information to a user;
a temperature sensor for sensing the temperature of the package, the temperature sensor being in communication with the dynamic display;
wherein the information is formed from changeable ink, such as printable ink, optical ink or e-ink; and
wherein the information includes a best-used-by date which is updated based on predefined parameters, including history of the temperature.

2. The compliance system of claim 1 further comprising a scanning device configured to communicate with the dynamic display.

3. The compliance system of claim 1 wherein the information is selected from the group consisting of expiration date, current time, current date, temperature information, time of next dose and a status indicator.

4. The system of claim 1 wherein the information is updated based on determining whether a combination of elapsed time and history of the temperature have set a condition such that contents of the package have expired.

5. The compliance system of claim 1 wherein the information is updated through direct communication with a server.

6. The compliance system of claim 1 wherein the information is updated by the scanning device.

7. The compliance system of claim 1 wherein the information is updated through a process run directly on the dynamic display.

8. The compliance system of claim 1 wherein the dynamic display further comprises an internal clock.

9. The compliance system of claim 1 wherein the ink has different colours to indicate different types of the information.

10. The compliance system of claim 1 wherein the scanning device is selected from the group consisting of a hand-held scanner, an RFID tag reader, a smart phone, a tablet, and a laptop.

11. The compliance system of claim 1 wherein the scanning device provides history and analytics of the information.

12. The compliance system of claim 1 wherein the scanning device provides the information shown on the dynamic display.

13. The compliance system of claim 1 wherein the scanning device provides current information selected from the group consisting of expiry date, current date, current time, date of next dose, time of next dose, date of last dose, number of doses taken, number of doses scheduled for the next time period, the temperature and amount of remaining medication.

14. The compliance system of claim 1 wherein the scanning device has multiple profiles for registering multiple users or multiple packages; and wherein the scanning device is configured to update each of the profiles individually or collectively as a whole.

15. The compliance system of claim 1 wherein the dynamic display is attached, adhered or connected to the package in a reusable or permanent manner.

16. The compliance system of claim 1 wherein the dynamic display further comprises a storage medium for tracking and storing the temperature of the package.

17. The compliance system of claim 16 wherein the dynamic display further comprises a transmitter and receiver for transmitting the stored temperature to a server, scanning device or elsewhere.

18. The compliance system of claim 1 wherein the dynamic display periodically polls the temperature sensor and the dynamic display further comprises a transmitter and receiver for transmitting the polled temperature to a server, scanning device or elsewhere.

19. The compliance system of claim 1 wherein the package is a smart package or smart cap containing medication.

20. The compliance system of claim 19 wherein the smart package or smart cap comprises a tag containing the temperature sensor.

21. The compliance system of claim 20 wherein the tag comprises a time tracking and dose monitoring system.

22. The compliance system of claim 20 wherein the smart package or smart cap has predefined parameters which allow the tag to run a process and determine expiration conditions of the medication; wherein the expiration conditions are communicated from the tag to the dynamic display to update the information.

23. The compliance system of claim 22 wherein the smart package or smart cap updates the information on the dynamic display between removal of dosages of medication.

24. The compliance system of claim 1 further comprising an indicator for a user to indicate each time a content of the package is removed, wherein the time and conditions of removal are recorded.

25. The compliance system of claim 1 wherein the smart cap or smart blister automatically senses removal of the contents of the package and records the time and conditions of removal.

26. The compliance system of claim 24 wherein the information is updated based on determining whether the time and conditions of removal have set a condition such that contents of the package have expired.

27. The compliance system of claim 1 further comprising a warning which alerts the user to expiry of the contents of the package.

28. A compliance system, comprising:
a scanning device which receives one or more health related activities as an input;
a slider for allotting a time interval;
a counter for allotting frequency;
a series of one or more clocks;
wherein the clocks set the time during a predetermined time period for performing one of the health related activities based on the frequency and the time interval.

29. The compliance system of claim 28, further comprising:
a code on a package;
wherein the scanning device scans the code, determines the contents of the package and inserts the contents as one of the health related activities.

30. The compliance system of claim 29 further comprising the compliance system of claim 1.

31. The compliance system of claim 29 wherein the scanning device sets a number of dosages and spacing per dose based on the code.

32. The compliance system of claim 28 wherein the health related activities are selected from the group consisting of exercise, food preparation, food consumption, meal times, medication expiry, medication dosage times, meditation, prayer, and other lifestyle related items.

33. The compliance system of claim 28 wherein the predetermined time period avoids overnight times.

34. The compliance system of claim 28 wherein the scanning device is selected from the group consisting of a hand-held scanner, an RFID tag reader, a smart phone, a tablet, and a laptop.

35. The compliance system of claim 28 wherein the scanning device is configured to communicate with an other device selected from the group consisting of a smart phone, tablet, a laptop and a computing device, wherein the other device receives the one or more health related activities as an input and the other device comprises the slider, the counter and the series of one or more clocks.

36. The compliance system of claim 28 further comprising an alarm for setting a reminder connected to one or more of the series of one or more clocks.

37. The compliance system of claim 28 wherein the alarm sends the reminder to the scanning device, to a cell phone as a text message, to an email address, or to a telephone call.

38. The compliance system of claim 28 further comprising an indicator for a user to indicate each time a health-related activity was completed.

39. The compliance system of claim 38 wherein the scanning device, other device or indicator stores, tracks and/or sends the completed health-related activities to a health practitioner in order to track compliance of the user.

40. The compliance system of claim 39 further comprising a reward for the compliance which satisfies minimum requirements.

41. The compliance system of claim 40 wherein the reward is selected from the group consisting of monetary, coupons, discounts and credits toward future purchases.

42. The compliance system of claim 28 wherein the time interval and the frequency are updated based on information received from the group consisting of a cloud, a server, a database, the scanning device or a health practitioner.

43. A system for monitoring a package, comprising:
a scanning device;
a dynamic display on the package and configured to communicate information with the scanning device;
wherein the scanning device sends the information to a server;
wherein the scanning device receives an update from the server; and
wherein the scanning device sends the update to the dynamic display and the dynamic display changes in response to the update.

44. The system of claim 43 wherein the server determines whether elapsed time and exposed temperature have led to conditions such that contents of the package should no longer be consumed.

45. A method comprising the steps of:
a. scanning a dynamic display on a package with a scanning device to obtain information;
b. communicating the information to a server with the scanning device;
c. processing the information by the server to determine whether a combination of elapsed time and exposed temperature have led to expiry of contents of a package;
d. communicating an update to the scanning device;
e. sending the update to the dynamic display from the scanning device; and
f. changing the information shown on the dynamic display based on the update.

46. The compliance system of claim 20 wherein the smart package or smart cap communicates with the scanning device or server to receive updates to the information.

47. The compliance system of claim 29, wherein the code is a QR code.

48. The compliance system of claim 1, further comprising a dynamic QR code containing encoded dose information for optical scanning by a scanning device.
